(19) United States
(12) Patent Application Publication
(10) Pub. No.: US 2015/0122264 A1
(43) Pub. Date: May 7, 2015

(54) CURVED DISTAL TIP FOR USE WITH MEDICAL TUBING AND METHOD FOR MAKING THE SAME

(71) Applicant: Covidien LP, Mansfield, MA (US)

(72) Inventors: Kieran M. Donlon, Co. Longford (IE); Carol Kiernan, Co. Westmeath (IE); Alan Finneran, Co. Offaly (IE); James P. Curley, Co. Offaly (IE); Emmet G. Bolger, Athlone (IE); Kamlesh S. Sethiya, Athlone (IE)

(21) Appl. No.: 14/529,852

(22) Filed: Oct. 31, 2014

Related U.S. Application Data
(60) Provisional application No. 61/898,969, filed on Nov. 1, 2013.

(51) Int. Cl. A61M 16/04; B29C 65/18

(52) U.S. Cl. CPC A61M 16/0465; B29C 65/18; A61M 2207/00; A61M 2207/10; B29L 2031/7548
USPC 128/207.14; 156/294

(57) ABSTRACT

A method of manufacturing a tracheal tube assembly including a cannula having a tapered distal tip is provided. The method includes providing a cannula having a distal end and inserting the distal end into a tapered melt mold. The method also includes applying heat to the tapered melt mold and the inserted distal end to form the tapered distal tip. The tapered distal tip may taper towards a distal end of the distal tip.

### CROSS-REFERENCE TO RELATED APPLICATIONS

This application claims the benefit of U.S. Provisional Application No. 61/898,969 filed Nov. 1, 2013, which is hereby incorporated by reference in its entirety for all purposes.

### BACKGROUND

The present disclosure relates generally to the field of tracheal tubes and, more particularly, to a tracheal tube including an outer cannula with a curved distal end.

This section is intended to introduce the reader to various aspects of art that may be related to various aspects of the present disclosure, which are described and/or claimed below. This discussion is believed to be helpful in providing the reader with background information to facilitate a better understanding of the various aspects of the present disclosure. Accordingly, it should be understood that these statements are to be read in this light, and not as admissions of prior art.

A wide variety of situations exist in which artificial ventilation of a patient may be desired. For short-term ventilation or during certain surgical procedures, endotracheal tubes may be inserted through the mouth to provide oxygen and other gasses to a patient. For other applications, particularly when longer-term intubation is anticipated, tracheostomy tubes may be preferred. Tracheostomy tubes are typically inserted through an incision made in the neck of the patient and through the trachea. A resulting stoma is formed between the tracheal rings below the vocal chords. The tracheostomy tube is then inserted through the opening. In general, two procedures are common for insertion of tracheostomy tubes, including a surgical procedure and a percutaneous technique.

One difficulty that arises in the use of tracheal tubes, and tracheostomy tubes in particular, is in comfortably inserting the tracheostomy tube into the patient. For example, the design of the tracheostomy tube, and in particular, the design of the distal end of the tracheostomy tube, may not allow for easy and comfortable insertion. There is a need, therefore, for improved tracheal tubes, and particularly for improved distal ends of tracheostomy tubes.

### BRIEF DESCRIPTION

This disclosure provides a novel tracheal tube that facilitates insertion into a patient's trachea. The tracheal tube may be a tube with a separate inner cannula and outer cannula. The outer cannula may be curved and/or flexible for ease of insertion and patient comfort. In particular, the outer cannula may include a longitudinal axis that is curved. The outer cannula may include a tapered distal tip. In some embodiments, the tapered distal tip may be formed using a melt mold (e.g., a melt shell) having a tapered end. For example, a distal end of the outer cannula may be inserted into a melt mold, and a portion of the distal end of the outer cannula may be melted using heat applied to the melt mold to form a tapered distal tip. The melt mold may include a longitudinal axis that follows the curve of the longitudinal axis of the outer cannula. That is, the melt mold and/or the opening defined by the melt mold may have the same curvature of the outer cannula. In some embodiments, the melt mold may define an opening that includes a first portion that may surround the distal end of the outer cannula when the outer cannula is inserted into the melt mold and may define a second portion that may extend past the distal end of the outer cannula. For example, an interior cavity of the melt mold may taper towards a distal end such that the cavity in the second portion is thinner than a wall of the outer cannula and such that the distal end of the outer cannula cannot advance into the second portion of the melt mold. Additionally, a wall of the second portion of the melt mold may be tapered. In particular, the wall of the second portion may be tapered such that the second portion is narrower in diameter at its distal end than its proximal end. In some embodiments, the second portion of the melt mold may be asymmetrical such that the angle or degree of taper varies about the circumference of the second portion. This may be advantageous to facilitate the insertion of the tracheal tube into the patient's trachea and may be particularly advantageous for tracheostomy tubes that are percutaneously inserted. Further, the tapered second portion may provide a more secure fit around, as well as a smoother transition between, an obturator and/or an introducer that may be used with the tracheal tube.

Thus, in accordance with a first aspect, a method of manufacturing a tracheal tube assembly includes providing a cannula having a cut distal end. The method also includes inserting the cut distal end into a melt mold. The melt mold includes an interior cavity configured to surround the cut distal end when inserted. Additionally, a portion of the interior cavity includes a tapered region that tapers the cavity to a terminus extending beyond the inserted cut distal end. The cavity at the terminus is thinner than a wall of the cannula such that the cut distal end cannot advance into the terminus of the interior cavity when inserted. Additionally, the method includes applying heat to the melt mold and the inserted cut distal end to melt a portion of the cut distal end into the terminus of the interior cavity to form a tapered distal tip.

In accordance with another aspect, a tracheal tube assembly includes a cannula including a distal end. The assembly further includes a melt tip disposed on the distal end of the outer cannula. The melt tip includes a tapered cavity that extends past the distal end of the outer cannula. The tapered region narrows away from the distal end.

Also disclosed herein is a melt mold for a tracheal tube. The melt mold includes a proximal end including a proximal opening of an interior cavity. The melt mold also includes a closed distal end. Additionally, the melt mold includes an indent formed by the closed distal end and extending into the interior cavity to define an interior surface of the interior cavity. A first gap between the interior surface and an exterior wall of the melt mold is configured to permit insertion of a tracheal tube end into the cavity. A second gap between the interior surface and the exterior wall of the melt mold is configured to be smaller than the tracheal tube end to prevent advancement of the tracheal tube end into the second gap. The second gap terminates at the closed distal end.

### BRIEF DESCRIPTION OF THE DRAWINGS

Various aspects of the disclosed techniques may become apparent upon reading the following detailed description and upon reference to the drawings in which:

FIG. 1 is a perspective view of a tracheal tube with an outer cannula and a tapered distal tip inserted into a patient in accordance with embodiments of the present disclosure;

FIG. 2 is an exploded perspective view of an outer cannula and a tapered melt mold in accordance with embodiments of the present disclosure;

FIG. 3 is a partial cross-sectional view of an outer cannula inserted into a tapered melt mold in accordance with embodiments of the present disclosure;

FIG. 4 is a perspective view of the tapered melt mold in accordance with embodiments of the present disclosure;

FIG. 5 is a cross-sectional view of a tapered melt mold in accordance with embodiments of the present disclosure;

FIG. 6 is a partial cross-sectional view of an assembled tracheal tube including an outer cannula and a tapered distal tip in accordance with embodiments of the present disclosure;

FIG. 7 is a process flow diagram of a method of manufacturing a tracheal tube including an outer cannula and a tapered melt mold in accordance with embodiments of the present disclosure; and

FIG. 8 is a perspective view of a tracheal tube assembly including an outer cannula with a tapered distal tip used in conjunction with an obturator.

### DETAILED DESCRIPTION OF SPECIFIC EMBODIMENTS

One or more specific embodiments of the present techniques will be described below. In an effort to provide a concise description of these embodiments, not all features of an actual implementation are described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

The tracheal tubes as provided herein may be disposable or reusable, capable of providing differential mechanical ventilation to either or both lungs, and capable of supporting all other functions of standard tracheal tubes (e.g. sealing, positive pressure generation, suctioning, irrigation, drug instillation, etc). The tracheal tubes can be used in conjunction with all acceptable auxiliary airway devices such as (e.g. heat and humidity conservers, mechanical ventilators, humidifiers, closed suction systems, scavengers, capnometers, oxygen analyzers, mass spectrometers, PEEP/CPAP devices, etc). Furthermore, although the embodiments of the present disclosure illustrated and described herein are discussed in the context of tracheal tubes such as tracheostomy tubes, it should be noted that presently contemplated embodiments may include a tracheal tube assembly including an outer cannula with a tapered distal end portion used in conjunction with other types of airway devices. For example, the disclosed embodiments may be used in conjunction with a single-lumen tube, an endotracheal tube, a double-lumen tube (e.g., a Broncho-Cath™ tube), a specialty tube, or any other airway device with a main ventilation lumen. Indeed, any device with a ventilation lumen designed for use in an airway of a patient may include an outer cannula with a tapered distal end portion as provided. As used herein, the term “tracheal tube” may include an endotracheal tube, a tracheostomy tube, a double-lumen tube, a bronchoblocking tube, a specialty tube, or any other airway device. Further, while the disclosed embodiments are shown with two cannulas (e.g., an inner cannula and an outer cannula), it should be understood that any of the disclosed embodiments may be used in conjunction with a single cannula.

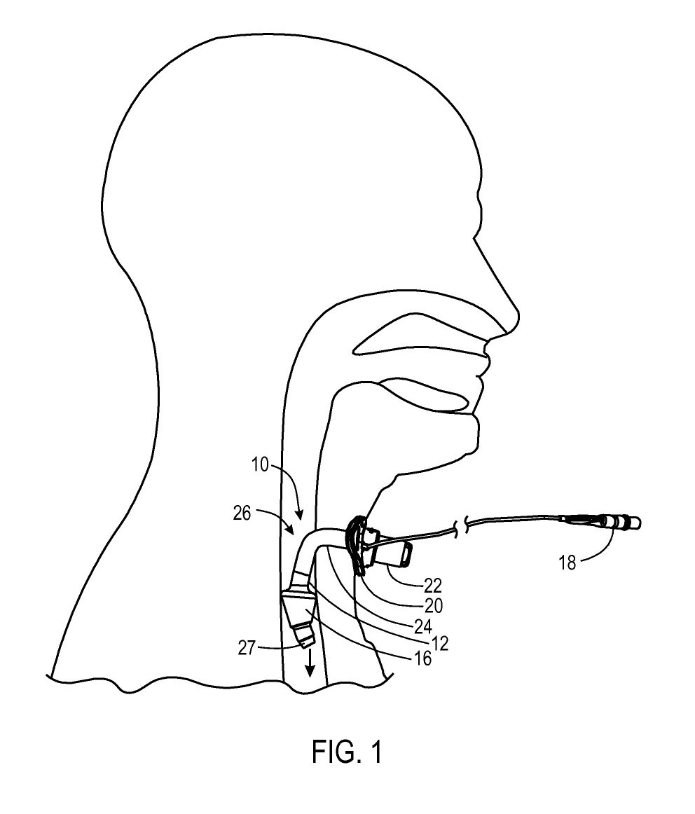

Turning now to the drawings, FIG. 1 is a perspective view of an exemplary tracheal tube 10 placed in a patient's airway in accordance with aspects of the present disclosure. The tracheal tube assembly 10 represented in the figures is a tracheostomy tube, although aspects of this disclosure could be applied to other tracheal tube structures, such as endotracheal tubes. The tracheal tube 10 includes an outer cannula 12 that defines a ventilation lumen and that facilitates the transfer of gases to and from the lungs. The tracheal tube 10 includes an inflatable cuff 16 disposed on the outer cannula 12. However, certain embodiments of the disclosure may be used in conjunction with cuffless tubes. A proximal end of the tracheal tube 12 may connect to upstream airway devices (e.g., a ventilator) via the appropriate medical tubing and/or connectors. In embodiments that include a cuff 16, a pilot balloon and inflation line assembly 18 is coupled to the cuff 16.

The outer cannula 12 is illustrated extending both distally as well as proximally from a flange member 20. A pair of side wings of the flange 20 extend laterally and serve to allow a strap or retaining member (not shown) to hold the tube assembly 10 in place on the patient. In one embodiment, apertures formed in each side of the flange member 20 allow the passage of such a retaining device. In many applications, the flange member 20 may be taped or sutured in place as well. In some embodiments, a proximal portion of the outer cannula 12 that is outside of the patient may form an outer cannula connector 22. The outer cannula connector 22 may receive an end region of an inner cannula and form a secure connection with the inner cannula. During intubation, the tracheal tube assembly 10 is placed through an opening formed in the neck and trachea of a patient and extending into the patient airway. In certain embodiments, the tracheal tube assembly 10 is curved to accommodate the curved tracheal passageway. For example, the outer cannula 12 may be curved in an unbiased state (i.e., outside the patient) such that an inner curve 24 is generally positioned on a ventral side of the patient while the outer curve 26 is positioned on the dorsal side of the patient when the tracheal tube assembly 10 is inserted in the patient. Further, the outer cannula 12 may include a tapered and/or curved distal tip 27 to facilitate insertion into the patient. As provided herein, the tapered distal tip 27 of the outer cannula 12 may be formed using a melt mold.

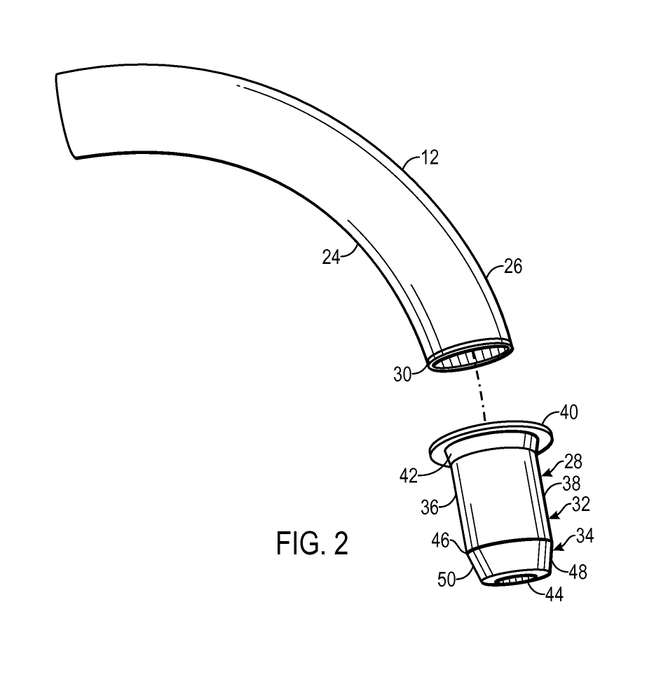

FIG. 2 is an exploded view of an embodiment of the outer cannula 12 and a melt mold 28. The outer cannula 12 and the melt mold 28 are separately formed and are separate structures. The outer cannula 12 and the melt mold 28 may be formed using any suitable manufacturing technique, such as extrusion, injection molding, compression molding, or casting molding. As will be described in more detail below, the tracheal tube assembly 10 may be manufactured or assembled by axially inserting a distal end 30 of the outer cannula 12 into the melt mold 28 and applying heat to the melt mold 28 and to the inserted distal end 30 of the outer cannula 12 to melt a portion of the inserted distal end 30 into the melt mold 28 to form the tapered distal tip 27.

The melt mold 28 may include a first portion 32 that may surround (e.g., fit about or mate with) the distal end 30 of the outer cannula 12 and a second portion 34 that may extend past the distal end 30 of the outer cannula 12 when the distal end 30 is inserted into the melt mold 28. In some embodiments, the first portion 32 of the melt mold 28 may be curved in an unbiased state (i.e., outside of the patient) similar to the outer cannula 12 to facilitate the insertion. In particular, similar to the outer cannula 12, the melt mold 28 may include an inner curve 36 and an outer curve 38 that are configured to align with (e.g., have the same degree of curvature as) the inner curve 24 and the outer curve 26 of the outer cannula, respectively. In certain embodiments, the curves 36 and 38 are present in the cavity formed by the first portion 32 and the second portion 34 and may or may not extend to the outer wall 39 of the melt mold 28. In this manner, the first portion 32 may follow the same curve (e.g., same alignment, complimentary curves) as the distal end 30 of the outer cannula 12. Accordingly, the insertion of the outer cannula 12 into the melt mold 28 may be directional such that proper insertion involves the inner curve 24 of the outer cannula 12 located proximate to or corresponding with the inner curve 36 of the melt mold 28. Similarly, the outer curve 26 of the outer cannula 12 may be located proximate to the outer curve 38 of the melt mold 28. Further, in embodiments in which the outer cannula 12 is tapered, for example having a smaller diameter at the distal end 30 than the proximal end, the first portion 32 of the melt mold 28 may include a complimentary taper.

The melt mold 28 may also include portions to facilitate the melting process. As illustrated, the melt mold 28 includes a collar portion 40 that may project radially outward from the melt mold 28. The collar portion 40 may be clamped to secure the melt mold 28 when heat is applied to the melt mold 28 and to the inserted distal end 30 of the outer cannula 12. In some embodiments, the melt mold 28 may also include a transition portion 42 disposed between the collar portion 40 and the first portion 32. As will be discussed in more detail below, the transition portion 42 may include a diameter that is larger than a diameter of the first portion 32 to facilitate the insertion of the distal end 30 of the outer cannula 12 into the melt mold 28.

As noted above, the melt mold 28 includes the second portion 34 that extends past the distal end 30 of the outer cannula 12 when the distal end 30 is inserted into the second portion 34. In some embodiments, the second portion 34 may be curved to align with the curve of the first portion 30 and/or the distal end 30 of the outer cannula. Additionally or alternatively, the second portion 34 may be tapered. For example, as illustrated, the second portion 34 may be tapered such that an outer diameter of a distal end 44 of the second portion 34 is less than an outer diameter of a proximal end 46 of the second portion 34. In some embodiments, the second portion 34 may be gradually tapered. In other embodiments, the second portion 34 may include a stepped taper. Additionally, as will be described in more detail below, the degree (e.g., angle) of taper of the second portion 34 may vary about the circumference of the second portion 34. For example, in some embodiments, an outer curve 48 of the second portion 34 may have greater a degree of taper than an inner curve 50. However, in other embodiments, the degree of taper of the inner curve 50 of the second portion 34 may be approximately equal to or less than the degree of taper of the outer curve 48 of the second portion 34.

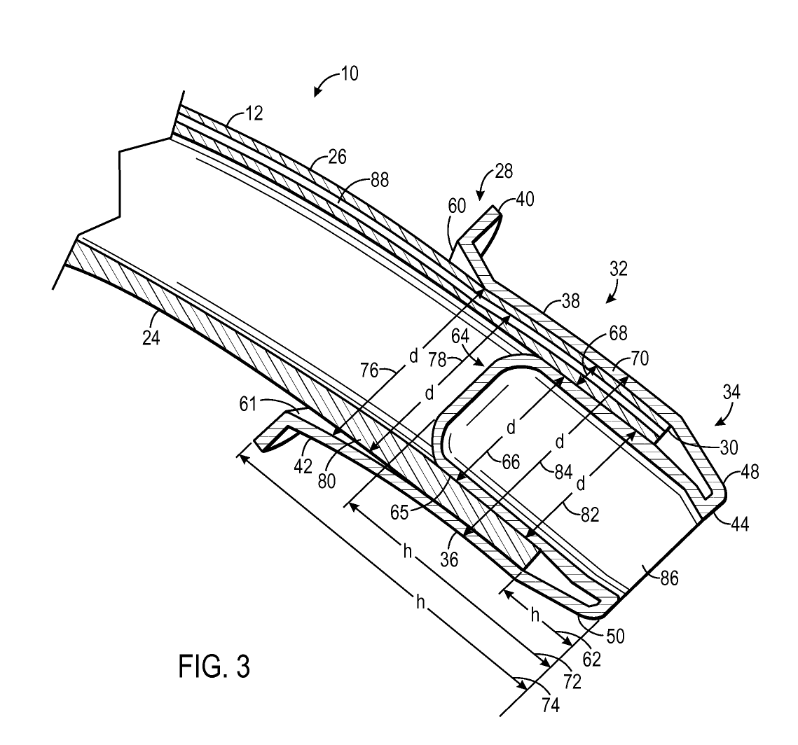

FIG. 3 is a cross-sectional view of the tracheal tube assembly 10 showing the outer cannula 12 inserted into the melt mold 28 prior to the melting process. The outer cannula 12 may be manually inserted into the melt mold 28 and/or automatically inserted, for example, via an automated procedure of manufacturing device. For example, the outer cannula 12 may be inserted by pushing the distal end 30 through a proximal opening 60 of an interior cavity 61 of the melt mold 28. The insertion may be complete when the distal end 30 of the outer cannula 12 is generally located at or near the second portion 34 of the melt mold 28. As illustrated, the distal end 30 of the outer cannula 12 terminates short of the distal end 44 (e.g., a closed distal end) of the melt mold 28.

The melt mold 28 may include one or more features to facilitate the placement of the distal end 30 of the outer cannula 12 at a distance 62 from the distal end 44 of the melt mold 28. Further, in some embodiments, the melt mold 28 may include an indent 64 that is formed by the distal end 44 and extends into the interior cavity 61 to define an interior surface 65 of the interior cavity 61. The indent 64 may be configured to be at least partially disposed within the outer cannula 12 (e.g., the distal end 30) when the outer cannula 12 is inserted in the interior cavity 61 of the melt mold 28. The indent 64 may define a generally cylindrical or barrel-shaped opening placed within the interior of the outer cannula 12. In some embodiments, an outer diameter 66 of the indent 64 may increase toward the distal end 44 of the melt mold 28 such that a space 68 (e.g., a gap) between an exterior wall 70 of the melt mold 28 and the interior surface 65 decreases toward the distal end 44 of the melt mold 28. In particular, the space 68 in the first portion 32 of the interior cavity 61 may have a width that is configured to permit insertion of the outer cannula 12 into the interior cavity, while the space 68 in the second portion 34 of the interior cavity 61 may be narrower than the width of the wall of the outer cannula 12 to prevent advancement of the outer cannula 12 into the second portion 34. Additionally, as noted above, the second portion 34 of the melt mold 28 may taper (e.g., narrow) toward the distal end 44 of the melt mold 28, which may also decrease the space 68 between the exterior wall 70 and the interior surface 61. The decreased width of the space 68 may facilitate the positioning of the outer cannula 12 at the distance 62 from the distal end 44 of the melt mold 28. As will be described in more detail below, positioning the outer cannula 12 at the distance 62 from the distal end 44 of the melt mold 28 may be desirable during the melting processing to enable the inserted distal end 30 of the outer cannula 12 to melt into the space 68 in the second portion 34 to form the tapered distal tip 27.

In addition to facilitating the desired placement of the outer cannula 12 into the melt mold 28, the indent 64 may also function to stabilize the melt mold 28 on the outer cannula 12 during a heating (e.g., melting) process. In particular, a height 72 of the indent 64 may be correlated with the stability. For example, a greater height 72 may yield a more secure fit between the melt mold 28 and the outer cannula 12 when the outer cannula 12 is inserted into the melt mold 28. In some embodiments, the height 72 of the indent 64 may be between approximately 20 percent and 100 percent, 30 percent and 90 percent, 40 percent and 80 percent, or 50 percent and 70 percent of a height 74 of the melt mold 28. In one embodiment, the height 72 of the indent 64 may be approximately two-thirds of the height 74 of the melt mold 28. For example, in some embodiments, the height 74 of the melt mold 28 may be between approximately 5 millimeters (mm) and 30 mm, 10 mm and 25 mm, or 15 mm and 20 mm. In one embodiment, the height 74 may be approximately 17 mm. Additionally, the height 72 of the indent 64 may be between approximately 5 mm and 25 mm, 8 mm and 20 mm, or 10 mm and 15 mm. In some embodiments, the height 72 of the indent 64 may be between approximately 10 mm and 12 mm.

The outer cannula 12 may have dimensions selected to fit easily through the stoma. In practice, a range of such tubes may be provided to accommodate the different contours and sizes of patients and patient airways. Such tube families may include tubes designed for neonatal and pediatric patients as well as for adults. By way of example only, the outer cannula 12 of the tube 10 may range from 4 mm to 16 mm. Accordingly, the melt mold 28 may be sized to correspond with an appropriate outer cannula 12 to facilitate the insertion of the outer cannula 12 within the melt mold 28.

Because the outer cannula 12 fits within the melt mold 28, at least a portion of the melt mold 28 features a larger inner diameter 76 relative to an outer diameter 78 of an inserted portion 80 of the outer cannula 12. As noted above, the melt mold 28 may be sized to correspond with the outer cannula 12. Accordingly, the inner diameter 76 may be selected based upon the size of the outer cannula 12. In certain embodiments, the inner diameter 76 may range from 5 mm to 20 mm.

Additionally, in embodiments in which the melt mold 28 includes the indent 64, the outer diameter 66 of the indent 64 may be less than an inner diameter 82 of the inserted portion 80 of the outer cannula 12 to facilitate the insertion of the outer cannula 12 between the outer wall 70 and the indent 64. Further, the space 68 between the outer wall 70 and the indent 64 in the first portion 32 of the melt mold 28 may be sized to correspond with the thickness of the walls of the outer cannula 12. For example, the walls of the inserted portion 80 of the outer cannula 12 may be about 1 millimeter (mm) thick. Accordingly, the space 68 in the first portion 32 of the melt mold 28 may be between about 1 mm to about 1.5 mm wide. Thus, an outer diameter 84 of the first portion 32 of the melt mold 28 may be at least 1 mm to 1.5 mm greater than the outer diameter 78 of the outer cannula 12.

The indent 64 may also define an exterior cavity 86 of the melt mold 28. The exterior cavity 86 may facilitate the melting process. In particular, heat may be applied to the exterior cavity 86, in addition to the exterior 70 of the melt mold 28, to facilitate the melting of the inserted portion 80 of the outer cannula 12 into the space 68 at the distal end 44 of the melt mold 28. Applying heat to the exterior cavity 86 may be desirable to decrease the time to melt the inserted portion 80. Melting the inserted portion 80 of the outer cannula 12 to form the tapered distal tip 27 may seal a lumen 88 (e.g., an inflation lumen to provide a gas to inflate the cuff 16) of the outer cannula 12. Thus, gases from the lumen 88 may be sealed off at the tapered distal tip 27 of the outer cannula 12.

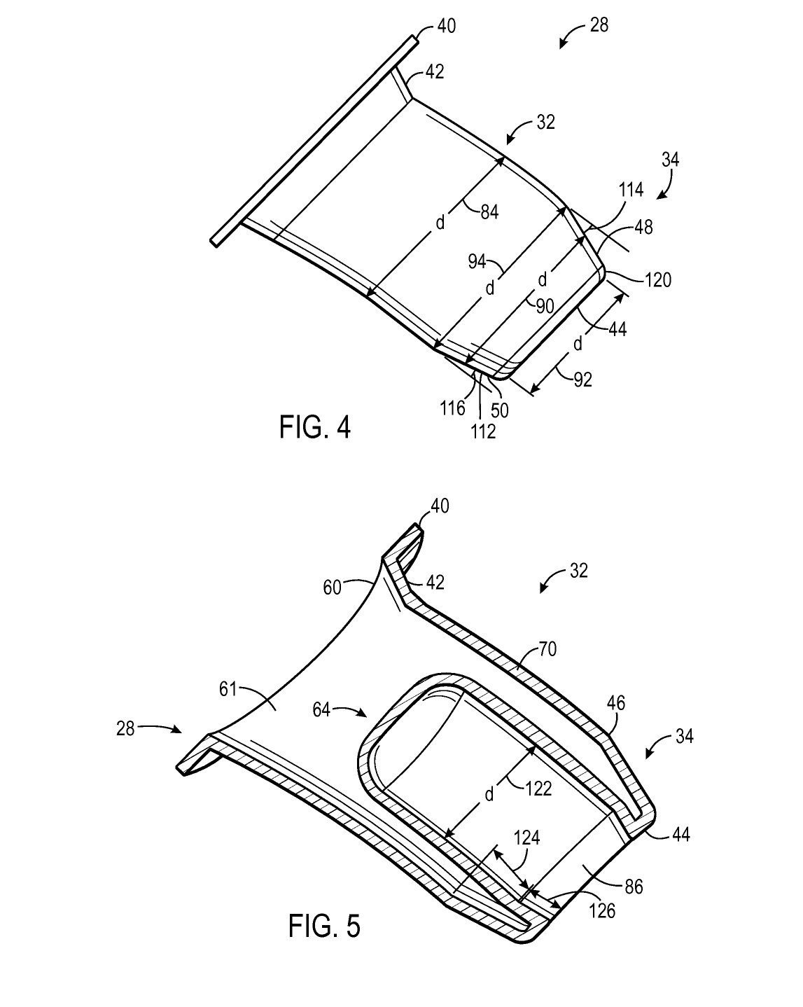

As noted above, the first portion 32 of the melt mold 28 may be tapered to facilitate the insertion of the distal end 30 of the outer cannula 12 into the melt mold 28. Further, as illustrated in FIG. 4, the melt mold 28 may be tapered from the first portion 32 to the second portion 34. In some embodiments, an outer diameter 90 of the second portion 34 of the melt mold 28 may be less than the outer diameter 84 of the first portion 32 of the melt mold 28. For example, in some embodiments, an outer diameter 92 of the distal end 44 of the melt mold 28 may be between approximately 5 percent to 95 percent, 25 percent to 90 percent, 45 percent to 85 percent, or 65 percent to 80 percent of the outer diameter 84 of the first portion 32 of the melt mold 28. In one embodiment, the outer diameter 92 of the distal end 44 of the melt mold 28 may be between approximately 70 to 75 percent of the outer diameter 84 of the first portion 32 of the melt mold 28. Further, the outer diameter 90 may decrease along the distance 62 of the second portion 34. For example, in certain embodiments, the outer diameter 92 of the distal end 44 may be between approximately 10 percent to 95 percent, 30 percent to 90 percent, 50 percent to 85 percent, or 70 percent to 80 percent of an outer diameter 94 of the proximal end 46 of the second portion 34 of the melt mold 28. Additionally, as noted above, the second portion 34 may be tapered. For example, the outer diameter 90 of the second portion 34 may be tapered. In some embodiments, the outer diameter 90 may be gradually tapered along the distance 62 with the outer diameter 90 of the distal end 44 narrower than the outer diameter 94 of the proximal end 46. For example, in one embodiment, the outer diameter 90 may continuously taper from the proximal end 46 to the distal end 44. Providing the melt mold 28 with the tapered second portion 34 may be desirable to create the tapered distal tip 27. That is, the inserted portion 80 of the outer cannula 12 may melt into the space 68 in the second portion 34 during the melting process and may take the form of the space 68 in the second portion 34.

Further, as noted above, the taper of the second portion 34 may vary about the circumference (e.g., about a rotational axis) of the second portion. In some embodiments, the degree (e.g., angle) of taper of an outer wall 112 of the second portion 34 may vary about the circumference of the outer wall 112 of the second portion 34. In some embodiments, the degree of taper of the outer wall 112 may continuously vary about the circumference of the outer wall 112. In particular, the outer curve 48 of the second portion 34, which may correspond to the outer curve 26 of the outer cannula 12, may have a first angle of taper 114. The inner curve 50, which may correspond to the inner curve 24 of the outer cannula 12, may have a second angle of taper 116. As illustrated, the first angle of taper 114 may be less than the second angle of taper 116. For example, the first angle of taper 114 may be between approximately 10 percent to 95 percent, 30 percent to 90 percent, 50 percent to 85 percent, or 70 percent to 80 percent of the second angle of taper 116. Alternatively, the first angle of taper 114 may be greater than the second angle of taper 116. Furthermore, in certain embodiments, the length of the outer curve 48 may be greater than the length of the inner curve 50. For example, the length of the inner curve 50 may be between approximately 10 to 100 percent, 50 to 95 percent, or 80 to 90 percent of the length of the outer curve 48.

Additionally, as illustrated in FIG. 4, the melt mold 28 may include a rounded end portion 120 at the distal end 44. In particular, the rounded end portion 120 may feature a different angle of taper than outer curve 48 and the inner curve 50. For example, the rounded end portion 120 may feature a greater angle of taper (e.g., a steeper taper) than the first angle of taper 114 of the outer curve 48 and the second angle of taper 116 of the inner curve 50. The rounded end portion 120 may be desirable to create a corresponding rounded end portion on the tapered distal tip 27 of the outer cannula 12. The rounded end portion of the tapered distal tip 27 may facilitate the insertion of the tracheal tube assembly 10 into the patient's trachea and may be more comfortable for the patient as compared to a straight (e.g., cornered) end. Further, the rounded end portion of the tapered distal tip 27, in addition to the curvature and the taper of the tapered distal tip 27, may provide a more secure fit between and/or a smoother transition between the tapered distal tip 27 and an introducer and/or an obturator that may be used in conjunction with the tracheal tube assembly 10. For example, in some embodiments, the degree of taper of the rounded end portion 120 and/or the curvature and taper of the second portion 34 may be selected such that the taper and/or curvature of the tapered distal tip 27 is continuous with the taper and/or curvature of an introducer and/or obturator.

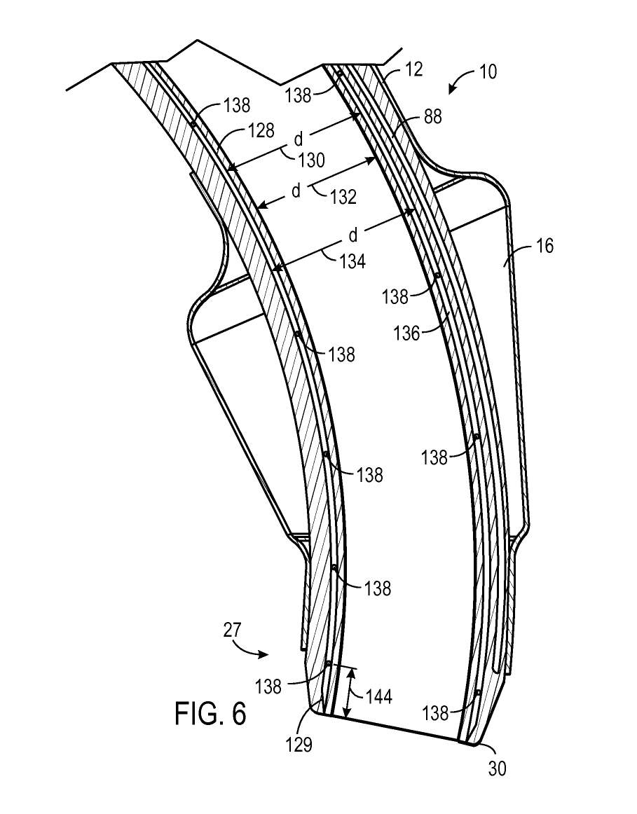

Furthermore, at least a portion of the exterior cavity 86 of the indent 64 of the melt mold 28 may be tapered. In particular, as illustrated in FIG. 5, an inner diameter 122 of the indent 64 may be tapered. In some embodiments, the inner diameter 122 of the second portion 34 may increase along a distance 124. In one embodiment, the inner diameter 122 may continuously taper along the distance 124. As noted above, this may be desirable to decrease the width of the space 68 to facilitate the placement of the outer cannula 12 (FIG. 3) within the melt mold 28 and to form the tapered distal tip 27. Further, the inner diameter 122 may decrease along a distance 126. In some embodiments, the inner diameter 122 may decrease gradually along the distance 126. For example, in one embodiment, the inner diameter 122 may continuously taper along the distance 126. Alternatively, the inner diameter 122 may include a stepped taper. The taper of the inner diameter 122 along the distance 126 may be desirable, because the tapered distal tip 27 that is formed during the melting process may take the shape of the melt mold 28 and, thus, may feature an inner diameter that tapers (e.g., narrows) toward the distal end of the tapered distal tip 27. An inner diameter of the tapered distal tip 27 that narrows along the distance 126 may facilitate the placement of an inner cannula within the outer cannula 12. In particular, an inner cannula may typically protrude from an outer cannula. However, it may be undesirable for the inner cannula to protrude from the outer cannula past a certain distance. Thus, the inner diameter 122 may be narrower at the distal end 44 than the proximal end 46 of the second portion 34 such that the formed tapered distal tip 27 may position an inner cannula within the outer cannula 12 such that the inner cannula does not protrude from the outer cannula 12 or protrudes from the outer cannula 12 by a selected distance (e.g., less than 2 mm or less than 1 mm). For example, FIG. 6 illustrates a cross-section of an embodiment of the assembled tracheal tube assembly 10 including the tapered distal tip 27, which is formed by melting the inserted portion 80 of the outer cannula 12 in the melt mold 28, and an inner cannula 128 disposed within the outer cannula 12. As illustrated, the inner cannula 128 does not protrude past the distal end 44 of the melt mold 28.

As shown in FIG. 6, the tapered distal tip 27 is shaped in the form of the interior cavity 61 (e.g., the space 68) of the melt mold 28. In particular, the outer diameter of the tapered distal tip 27 may decrease toward the distal end 30 of the outer cannula 12. Thus, the tapered distal tip 27 may narrow toward the distal end 30 and at least a portion of the tapered distal tip 27 may be narrower than other portions of the outer cannula 12. Additionally, in some embodiments, an end portion 129 of the tapered distal tip 27 may feature a decreasing inner diameter, which, as described above, may facilitate the positioning of the inner cannula 128 within the outer cannula 12. Further, as noted above, the end portion 129 of the tapered distal tip 27 may be rounded to facilitate insertion of the tracheal tube assembly 10 into the patient's trachea and to minimize discomfort to the patient.

An outer diameter 130 of the inner cannula 128 may be selected to allow sufficient air flow while also fitting comfortably within the outer cannula 12 and allowing for appropriate insertion force. An inner diameter 132 of the inner cannula 128 may be less than the outer diameter 130 by the thickness of the walls of the inner cannula 128. For example, an inner cannula 128 sized to 6.5 mm may have an outer diameter 130 of about 6.5 mm and an inner diameter 132 of about 5.5 mm. In such an embodiment, the inner cannula walls are about 1 mm thick in the inserted portion of the inner cannula 128 (e.g., in portions distal of the outer cannula connector 22). Similarly, a 10 mm inner cannula 128 may have an inner diameter of about 9 mm. Accordingly, tubes sized to 6.5 mm, 7.0 mm, 7.5 mm, 8.0 mm, 8.5 mm, 9.0 mm, or 10 mm may feature smaller inner diameters that define the airflow passage.

Further, an inner diameter 134 of the outer cannula 12 may be selected to allow for the inner cannula 128 to be easily inserted into the outer cannula 12. In particular, the inner diameter 134 of the outer cannula 12 may be slightly larger than the outer diameter 130 of the inner cannula 128 to facilitate the insertion of the inner cannula 128 into the outer cannula 12. For example, the inner diameter 134 of the outer cannula 12 may be larger than the outer diameter 130 of the inner cannula 128 by approximately 1 mm, 2 mm, 3 mm, 4 mm, 5 mm, or any other suitable distance. However, the difference in the inner diameter 134 of the outer cannula 12 and the outer diameter 130 of the inner cannula 128 may result in a gap 136 between the outer cannula 12 and the inner cannula 128 when the inner cannula 128 is inserted into the outer cannula 12. However, secretions (e.g., mucus) may accumulate inside of the gap 136, which may increase the patient's work of breathing. Furthermore, the secretions may cause the inner cannula 128 to become stuck to (e.g., adhere to) the outer cannula 12, which may cause a caregiver to apply a greater force to remove the inner cannula 128 from the outer cannula 12 (e.g., for cleaning or replacement). Additionally, the gap 136 may be undesirable because air that passes through the gap 136 may increase loss of air during ventilation, which may lead to inadequate ventilation of the patient. Additionally, the gap 136 may result in a whistling noise as the patient breathes, which may adversely affect the clarity of the patient's speech.

To mitigate the occurrence and/or severity of issues that may result from the gap 136, such as those described above, the tracheal tube assembly 10 may include features to facilitate that may seal the gap 136 between the inner cannula 128 and the outer cannula 12. For example, the tracheal tube assembly 10 may include one or more inflation devices 138. The one or more inflation devices 138 may include an inflatable bladder (e.g., a balloon), which may be configured to be inflated by any suitable inflation medium, such as a gas (e.g., air, nitrogen, or any other suitable gas) or a liquid. In particular, the system 10 may include an inflation system (not shown) configured to deliver the inflation medium to the one or more inflation devices 138. In certain embodiments, the inflation system may deliver the inflation medium to the inflation devices 138 via a lumen 140 of the inner cannula 128. Additionally or alternatively, the inflation system may deliver the inflation medium to the inflation devices 138 via a lumen of the outer cannula 12. In one embodiment, the inflation system may be coupled to a wall of the inner cannula 128 and/or a wall of the outer cannula 12. The inflation system may be configured to deliver an amount of the inflation medium suitable to cause the inflation devices 138 to seal (e.g., fill or block) the gap 136 between the inner cannula 128 and the outer cannula 12.

The inflation devices 138 may be formed in any suitable shape. For example, the inflation devices 138 may be spherical, tapered, barrel-shaped, rectangular, or the like. Additionally, each inflation device 138 may include a single inflatable bladder (e.g., balloon) or may include more than one inflatable bladder. The inflation devices 138 may be manufactured from any suitable polymer material with low to high wall thickness. By way of example, the inflation devices 138 may be formed from polyethylene teraphthalate (PETP), low-density polyethylene (LDPE), polyvinyl chloride (PVC), silicone, neoprene, polysioprene, polyurethane, or any combination thereof.

The inflation devices 138 may be disposed in the gap 136 in any location along the length of the tracheal tube assembly 10. The inflation devices 138 may be coupled in or on a wall of the inner cannula 128 or a wall of the outer cannula 12. In some embodiments, the inflation devices 138 may be secured to the inner cannula 128 or the outer cannula 12 via an adhesive. Further, the tracheal tube assembly 10 may include one inflation device 138 at a particular location along the length of the tracheal tube assembly 10 (e.g., at distance 144), or may include two or more inflation devices 138 at the same position (e.g., the distance 144) that are disposed about the circumference of the inner cannula 128. Furthermore, in one embodiment, the inflation device 138 may form a ring and may be disposed around the circumference of the inner cannula 128. Additionally, the tracheal tube assembly 10 may include any suitable number of inflation devices 138.

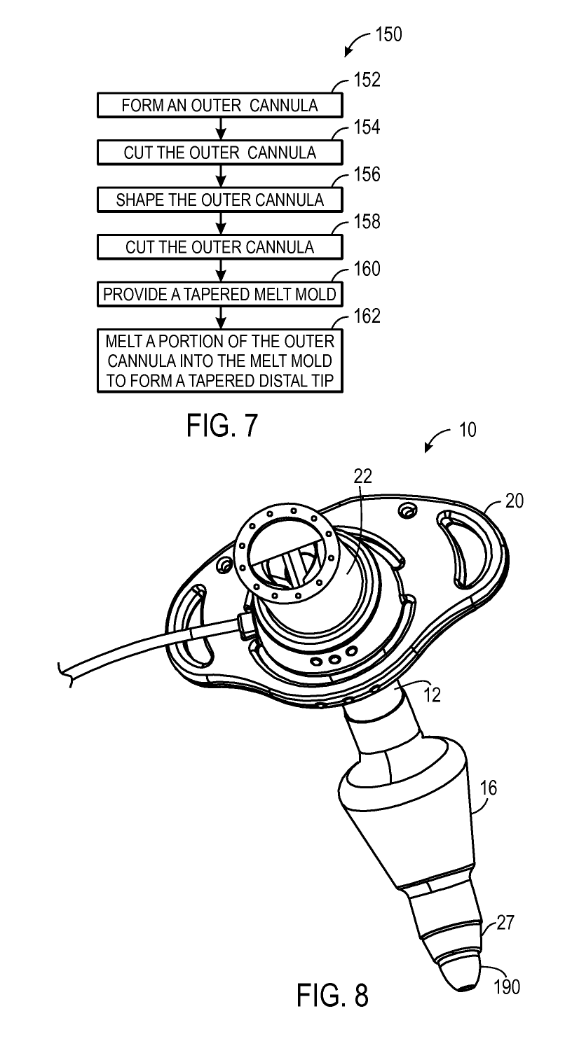

With the foregoing in mind, methods of manufacturing the embodiments of the tracheal tube assembly 10, as described above with respect to FIGS. 1-6, are also contemplated. For example, FIG. 7 illustrates an embodiment of a method 150 for manufacturing a tracheal tube assembly, such as the tracheal tube assembly 10. The method 150 may include forming a cannula (e.g., the outer cannula 12) (block 152). The outer cannula 12 may be formed using any suitable material or combination of materials, such as, for example, polyethylene (e.g., low density polyethylene), polypropylene, PTFE, expandable PTFE, polyvinyl chloride (PVC), PEBAX silicone, polyurethane, thermoplastic elastomers, polycarbonate plastic, silicon, or acrylonitrile butadiene styrene (ABS). In certain embodiments, the outer cannula 12 may be extruded. Extrusion may be desirable in some embodiments to manufacture the outer cannula 12 with one or more integral lumens (e.g., conduits). For example, the outer cannula 12 may be extruded to include a main respiratory lumen, an inflation lumen (e.g., the lumen 88), a suction lumen, and/or the lumen 140 for delivering an inflation medium to the one or more inflation devices 138. However, it should be noted that other manufacturing techniques may be utilized to form the outer cannula 12. For example, the outer cannula 12 may be molded, overmolded, two shot molded, computer numerical control (CNC) machined, milled, or otherwise formed into the desired shape.

The method 150 may also include cutting the outer cannula 12 (block 154) to a desired length. In one embodiment, the outer cannula 12 may be cut to a length of approximately 15 mm. The method 150 may also include shaping the outer cannula 12 (block 156). As noted above, it may be desirable for the outer cannula 12 to be curved in an unbiased state (i.e., outside the patient) such that the inner curve 24 is generally positioned on a ventral side of the patient while the outer curve 26 is positioned on the dorsal side of the patient when the tracheal tube assembly 10 is inserted in the patient. Accordingly, the outer cannula 12 may be shaped (e.g., bent or angled) into the desired curvature. For example, the outer cannula 12 may be placed on a mandrel having the desired curvature, and the outer cannula 12 may be heated (e.g., baked) to a temperature sufficient to set the curvature. In one embodiment, the outer cannula 12 may be heated for approximately seven minutes. After heating the outer cannula 12, the outer cannula 12 may be chilled, which may also facilitate setting the curvature of the outer cannula 12. In one embodiment, the outer cannula 12 may be chilled for approximately ten minutes.

The method 150 may include cutting the outer cannula 12 (block 158) to a second desired length. In certain embodiments, the second desired length of the outer cannula 12 may be based upon the extruded diameter of the outer cannula 12. Additionally, in some embodiments, the distal end 30 of the outer cannula 12 may be cut on an angle to create a beveled distal end.

Further, the method 150 may include providing a curved and/or tapered melt mold (e.g., the melt mold 28) (block 160). The melt mold 28 may be formed using any suitable material or combination of materials, such as, for example, stainless steel, iron, polyethylene (e.g., low density polyethylene), polypropylene, PTFE, expandable PTFE, polyvinyl chloride (PVC), PEBAX silicone, polyurethane, thermoplastic elastomers, polycarbonate plastic, silicon, or acrylonitrile butadiene styrene (ABS). In certain embodiments, the melt mold 28 may be manufactured using a material having a high melting point (e.g., higher than the melting point of the outer cannula 12). Furthermore, the melt mold 28 may be manufactured using any suitable technique. For example, the melt mold 28 may be molded, overmolded, two shot molded, computer numerical control (CNC) machined, milled, or otherwise formed into the desired shape, as described above with respect to FIGS. 1-6.

In some embodiments, the melt mold 28 may be disposed within, integrated within, or otherwise secured to a melt mold manufacturing apparatus. As used herein, a melt mold manufacturing apparatus may be any suitable device configured to apply heat to the melt mold 28 and/or the outer cannula 12 to melt a portion of the outer cannula 12 into the melt mold 28. In one embodiment, the melt mold 28 may be disposed within (e.g., embedded into) a platform of a melt mold manufacturing apparatus. Further, the platform may be coupled to a source of heat that may be configured to deliver heat to the melt mold 28 and/or the outer cannula 12 when the outer cannula 12 is inserted into the melt mold 28.

The method 150 may also include melting a portion of the outer cannula 12 into the melt mold 28 to form the tapered distal tip 27 (block 162). For example, the outer cannula 12 may be placed on a curved pin or mandrel and the melt mold 28 may be positioned about the distal end 30 of the outer cannula 12. In other embodiments, the outer cannula 12 may be placed on a curved pin or mandrel that may be configured to insert the outer cannula 12 into the melt mold 28. For example, the curved pin or mandrel may be secured to a movable platform of the melt mold manufacturing apparatus that is configured to move and/or rotate the curved pin or mandrel such that the curved pin or mandrel and the outer cannula 12, if placed on the curved pin or mandrel, is inserted into the melt mold 28.

As noted above, melt mold 28 may be directionally shaped such that the outer cannula 12 may enter the melt mold 28 in only one direction or orientation. At least a portion of the inserted portion 80 of the outer cannula 12 may be melted into the melt mold 28. For example, heat may be applied to the exterior of the melt mold 28 and/or the exterior cavity 86 of the indent 64 of the melt mold 28 when the inserted portion 80 is inserted in the melt mold 28 to melt the inserted portion 80 of the outer cannula 12. In some embodiments, pressurized gas may be applied to the melt mold 28 and/or the outer cannula 12 to facilitate the expansion of the melted portion of the outer cannula 12 into the melt mold 28. The melted inserted portion 80 of the outer cannula 12 may be cooled (e.g., chilled) inside of the melt mold 28. The outer cannula 12 may be removed from the melt mold 28, or vice versa, after cooling. As noted above, melting the inserted portion 80 of the outer cannula 12 may cause the inserted portion 80 of the outer cannula 12 to take the shape of the interior cavity 61 of the melt mold 28. Thus, the inserted portion 80 of the outer cannula 12 may be formed into the tapered distal tip 27. This may provide a tracheal tube assembly 10 that is more comfortable for the patient than tracheal tubes having straight tips (e.g., not curved) with straight, blunt ends. Further, providing the tapered distal tip 27 that is continuous with the outer cannula 12 may facilitate the insertion and positioning of the tracheal tube assembly 10 into the patient.

It is envisioned that the tracheal tube assembly 10 as provided herein may be provided as an assembly and/or as a kit. A kit may include a packaging that encloses an inner cannula 128 sized for an outer cannula 12, which may include the tapered distal tip 27 manufactured using the melt mold 28, an affixed outer cannula connector 22 and flange member 20. The kit may also include a neck strap for retaining the tracheal tube 10 in place. The kit may also include an obturator 190, shown in FIG. 8. Other components of the kit may include a cap configured to be placed on a proximal end 34 while the obturator 190 is in use and that may be part of the obturator 190. The tracheal tube assembly 10 components (e.g., outer cannula 12, flange member 20, outer cannula connector 28, cuff 16, and pilot balloon assembly 18) may be assembled prior to in situ assembly of the inner cannula 12 into the outer cannula 14. Indeed, the user or clinician may perform final assembly of the tracheal tube assembly 10 by selecting a desired inner cannula 128 from a selection of inner cannulas and then inserting the inner cannula 128 into the outer cannula 12 prior to intubation. Thus assembled, the tracheal tube assembly 10 may then be inserted into the patient's trachea.

While the disclosure may be susceptible to various modifications and alternative forms, specific embodiments have been shown by way of example in the drawings and have been described in detail herein. However, it should be understood that the embodiments provided herein are not intended to be limited to the particular forms disclosed. Indeed, the disclosed embodiments may not only be applied to airway devices, but these techniques may also be utilized for connections between inner and outer conduits for other types of medical devices and medical connective tubing. Rather, the various embodiments may cover all modifications, equivalents, and alternatives falling within the spirit and scope of the disclosure as defined by the following appended claims.

What is claimed is:

1. A method of manufacturing a tracheal tube assembly, comprising:
providing a cannula having a cut distal end;
inserting the cut distal end into a melt mold, wherein the melt mold comprises an interior cavity configured to surround the cut distal end when inserted and wherein a portion of the interior cavity comprises a tapered region that tapers the cavity to a terminus extending beyond the inserted cut distal end, wherein the cavity at the terminus is thinner than a wall of the cannula such that the cut distal end cannot advance into the terminus of the interior cavity when inserted; and
applying heat to the melt mold and the inserted cut distal end to melt a portion of the cut distal end into the terminus of the interior cavity to form a tapered distal tip.

2. The method of claim 1, comprising cooling the melt mold and the tapered distal tip and removing the melt mold from the tapered distal tip.

3. The method of claim 1, wherein an angle of a taper of the tapered region varies about the circumference of the tapered region.

4. The method of claim 1, wherein an interior diameter at the terminus is narrower than an interior diameter of the cut distal end such that the tapered distal tip is at least in part narrower than other portions of the cannula.

5. The method of claim 1, wherein the cannula comprises a lumen open to the cut distal end and wherein applying heat to the inserted cut distal end seals the lumen when forming the tapered distal tip.

6. The method of claim 1, wherein an exterior of the melt mold comprises an exterior forming an opening at a distal end of the melt mold and terminating in a closed surface at a location closer to a proximal end of the melt mold than the distal end.

7. The method of claim 1, wherein applying heat to the melt mold comprises applying heat to a surface of the exterior cavity.

8. The method of claim 1, wherein the interior cavity is open at a proximal end of the melt mold and closed at the terminus.

9. The method of claim 1, wherein the cannula is curved along its length to form an inside curve and an outside curve and wherein the interior cavity of the melt mold conforms to the inside curve and the outside curve.

10. The method of claim 9, comprising inserting a curved mandrel inside the cannula and heating the cannula and the curved mandrel to form the inside curve and the outside curve.

11. A tracheal tube assembly comprising:
a cannula comprising a distal end; and
a melt tip disposed on the distal end of the outer cannula, wherein the melt tip comprises a tapered cavity that extends past the distal end of the outer cannula, wherein the tapered region narrows away from the distal end.

12. The tracheal tube assembly of claim 11, wherein the tracheal tube comprises a tracheostomy tube.

13. The tracheal tube assembly of claim 11, wherein an angle of taper of the tapered region varies about the circumference of the tapered region.

14. The tracheal tube assembly of claim 11, wherein the outer cannula comprises a lumen formed in a wall of the outer cannula.

15. The tracheal tube assembly of claim 11, wherein the cannula and at least a portion of the distal tip comprise a same degree of curvature.

16. The tracheal tube assembly of claim 11, comprising an inner cannula configured to be inserted into the cannula.

17. A melt mold for a tracheal tube, comprising:
a proximal end comprising a proximal opening of an interior cavity;
a closed distal end; and
an indent formed by the closed distal end and extending into the interior cavity to define an interior surface of the interior cavity, wherein a first gap between the interior surface and an exterior wall of the melt mold is configured to permit insertion of a tracheal tube end into the cavity and wherein a second gap between the interior surface and the exterior wall of the melt mold is configured to be smaller than the tracheal tube end to prevent advancement of the tracheal tube end into the second gap, wherein the second gap terminates at the closed distal end.

18. The melt mold of claim 17, wherein the second gap narrows towards the closed distal end.

19. The melt mold of claim 17, wherein the indent forms an exterior cavity, and wherein the exterior cavity is narrower at the closed distal end.

20. The melt mold of claim 17, wherein the indent terminates at a location closer to the proximal end than the distal end.
